(19) United States
(12) Patent Application Publication
(10) Pub. No.: US 2025/0104569 A1
(43) Pub. Date: Mar. 27, 2025

(54) Game Training for Neuroplasticity for Healthy Aging

(71) Applicant: Board of Regents, The University of Texas System, Austin, TX (US)

(72) Inventors: Chandramallika Basak, Dallas, TX (US); Evan T. Smith, Dallas, TX (US)

(21) Appl. No.: 18/896,352

(22) Filed: Sep. 25, 2024

Related U.S. Application Data
(60) Provisional application No. 63/585,176, filed on Sep. 25, 2023.

(51) Int. Cl. G09B 5/02; G16H 10/20

(52) U.S. Cl. CPC G09B 5/02; G16H 10/20

(57) ABSTRACT

A computer-implemented neurocognitive training game is provided. The game comprises first presenting, in a user interface, a number of questions regarding different health parameters of a user within a previous 24 hour period. Responsive to entry of answers to all of the questions, a number of trials are presented in the user interface. Each trial has a specified response time window and comprises a number of images displayed within spatially distinct contexts. A user score is generated based on the correct identification of the images in the trials by the user. The difficulty and the response time window is adjusted for the trials according to the user score.

### CROSS-REFERENCE TO RELATED APPLICATION

This application claims the benefit of U.S. Provisional Patent Application Ser. No. 63/585,176, filed Sep. 25, 2023, and entitled “Game Training for Neuroplasticity,” which is incorporated herein by reference in its entirety.

### STATEMENT OF GOVERNMENT SUPPORT

This invention was made with government support under Grant No. R56AG060052 awarded by the National Institutes of Health. The government has certain rights in the invention.

### BACKGROUND INFORMATION

### 1. Field

The field of the invention is a game for training working memory, cognitive control and other complex cognitive functions in a human brain, especially in the aged and for those at risk for Alzheimer's disease.

### 2. Background

It is estimated that there are more than 6 million Americans diagnosed with Alzheimer's disease (AD) in 2021, the most common form of dementia, and this number is expected to double every 20 years, to nearly 14 million in 2050. Moreover, we are also going to see an unprecedented increase in older adults in our population with the last baby boomer reaching 65 years of age in 2030. People age 65+ represented 17% of the population in the year 2020. This is expected to grow to 22% by 2040. The prevalence of AD and the associated costs to individuals and to society continue to grow as our population ages without an effective therapeutic approach. During the last twenty years, only seven drugs for treating AD were approved by the U.S. Food and Drug Administration (FDA). None of these drugs significantly impact AD neuropathology, and at best currently available drug treatments only slow symptom progression for a limited time. Against this paucity of treatment options, one of our greatest contemporary challenges is to elucidate and deploy more effective strategies for changing the course of cognitive aging to more reliably assure that brain spans more closely match our growing life spans. The most impact on reducing the long-term burden of cognitive decline on the individual and communities is from intervening early during preclinical periods before overt cognitive impairment.

### SUMMARY

An illustrative embodiment provides a computer-implemented neurocognitive training game comprising presenting, in a user interface, a number of questions regarding different health parameters of a user within a previous 24 hour period. Responsive to entry of answers to all of the questions, a number of trials are presented in the user interface. Each trial has a specified response time window and comprises a number of images displayed within spatially distinct contexts. A user score is generated based on the correct identification of the images in the trials by the user. The difficulty and the response time window is adjusted for the trials according to the user score.

The features and functions can be achieved independently in various embodiments of the present disclosure or may be combined in yet other embodiments in which further details can be seen with reference to the following description and drawings.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 depicts the unified modeling language of the close-loop game in accordance with an illustrative embodiment;

FIG. 2 depicts a login page and daily health screening of mood in accordance with an illustrative embodiment;

FIG. 3 depicts a single trial from the game with contexts and reward context in accordance with an illustrative embodiment;

FIG. 4A depicts a graph of target distribution in accordance with an illustrative embodiment;

FIG. 4B depicts a graph of lure distribution in accordance with an illustrative embodiment;

FIG. 5 depicts possible outcomes in accordance with an illustrative embodiment;

FIG. 6 shows a number of different versions of the game that are available in accordance with an illustrative embodiment;

FIG. 7 depicts and example trial wherein the trees turn to fall colors in the “I” version of the game in accordance with an illustrative embodiment;

FIG. 8 depicts a graph of autocorrelation of accuracy in accordance with an illustrative embodiment;

FIG. 9 depicts a graph of lag on average response time and accuracy in accordance with an illustrative embodiment;

FIG. 10 depicts a graph of frontal region deactivation in older adults in accordance with an illustrative embodiment;

FIG. 11 depicts imagining showing maintenance of left lateral parietal and frontal gray matter volumes and cortical thickness of left parietal regions maintenance of left lateral parietal and frontal gray matter volumes and cortical thickness of left parietal regions; and

FIG. 12 depicts a graph illustrating structure-cognition correlations in accordance with an illustrative embodiment.

### DETAILED DESCRIPTION

The illustrative embodiments recognize and take into account that the most impact on reducing the long-term burden of cognitive decline on the individual and communities is from intervening early during preclinical periods before overt cognitive impairment. Intervening during the pre-clinical period is important because the pathological processes that lead to AD and other forms of dementia begin years before its diagnosis. The long pre-clinical, healthy aging phase of AD therefore provides a key opportunity for introducing interventions that optimize cognitive functions and potentially prevent AD. In order to maintain the quality of life and decrease the medical burden of a rapidly aging society, it is important that we develop principles of neurocognitive optimization that will maintain cognitive functions into very old age and potentially delay the onset of AD diagnosis. Meta-analysis suggests that cognitive training of executive control and working memory holds promise for improving age-sensitive cognitive skills in both healthy aging and in adults diagnosed with Mild Cognitive Impairment, a pre-clinical dementia stage.

However, no training so far has simultaneously trained these abilities in an individualized adaptive manner, particularly the unpredictably cued working memory updating training. Research indicates greatest neurocognitive deficits in older adults are during memory updating, esp. for unpredictable cues.

Older adults aged 65 years or older show significant declines in many cognitive abilities, especially in working memory, executive functions, information processing speed, reasoning and episodic memory. Importantly, adults with pre-clinical dementia, such as those diagnosed with Mild Cognitive impairment (MCI), show greatest declines in executive functions and cognitive tasks that are related to frontal-parietal brain functioning. Working memory (the ability to coordinate between multiple items) and attentional control (the ability to exert control to anticipatory predictable events, and respond flexibly to unpredictable events) are fundamental to nearly all complex cognitive tasks, and show marked age-related detriments. Several studies in healthy older adults show that training on predictably-cued working memory updating (e.g., the n-back task) improves working memory capacity (a near ability), with some evidence that it can increase frontal-parietal activity in the trained ability and white matter integrity. However, benefits to other cognitive skills (far abilities) are small or nonexistent with these n-back tasks where cues appear in a predictable sequence.

There is obviously a great need for an intervention program that: (1) more effectively targets the neurocognitive deficits that slowly compromise brain function and cognition in older adults; (2) are personalized to the user; (3) carry negligible side effects; (4) are scalable, affordable, and deliverable into every American community and home; (5) do not put older adults at risk through use of online game training which can lead to targeted frauds on older adults that are increasing over last few years; and (5) have real-world functional efficacy demonstrable immediately and over the longer term. Our present aims to fulfill all these objectives. The BirdWatch Game has all these advantages as well as evidence that this program could be implemented on stand-alone inexpensive Android tablets with no internet connectivity to older adults (65-80 years of age) during COVID-19 period effectively from their home, where adherence to the 20 hour/8-week training program was high (average training hours: ˜ 18 hours) in spite of the mental health impacts of COVID-19.

The illustrative embodiments provide a dual-pronged closed-loop, individualized adaptive, evidence based neurocognitive training program that simultaneously trains focusing attention through unpredictably cued memory updating training and working memory capacity by adaptively improving memory discriminability and processing time. This closed-loop individualized adaptive program first targets improving memory discriminability and then improves proceeding speed. The main premise of the brain training program, called BirdWatch Game is to simultaneously train fundamental age-sensitive cognitive skills of working memory and cognitive control. The BirdWatch Game uses engaging game-based simulations, where attentional control demands in working memory training are systematically increased.

Our BirdWatch Game is based on the principle of cognitive optimization through unpredictably cueing memory updating, compared to existing training programs that have used predictable memory updating (e.g., Dual N-Back) or used unpredictable cues but without any working memory updating demands (e.g., Neuroracer, studies using task-switching or dual task paradigms).

The present invention builds on this past research on the n-back task, where, importantly, the cues were always predictable. We take a new theory-driven approach in this disclosure to working memory training based on the promising findings from the PI's laboratory, where unpredictable cues in working memory, which require greater attentional control than predictable cues, resulted in greater gains in a task of episodic memory (a far ability) in older adults. However, this unpredictable memory training was not gamified, was not individualized adaptive and was an open-loop system. The abilities were also measured using a single representative task, so it is not clear if the unpredictable attentional control during working memory training can enhance broad cognition or is limited to few tasks. Also, it is not known if the unpredictable working memory training enhances brain structures and functions that degrade not only with aging but are early markers of Alzheimer's Disease (AD).

The concepts of the present game are disclosed in relation to an embodiment called the bird watch game. However, it should be understood that the disclosed concepts may be practiced in any number of suitable game environments and with suitable graphics, and not limited to a bird watching environment, but in any environment that may implement the disclosed methods for training working memory, cognitive control and other complex cognitive functions in a human brain.

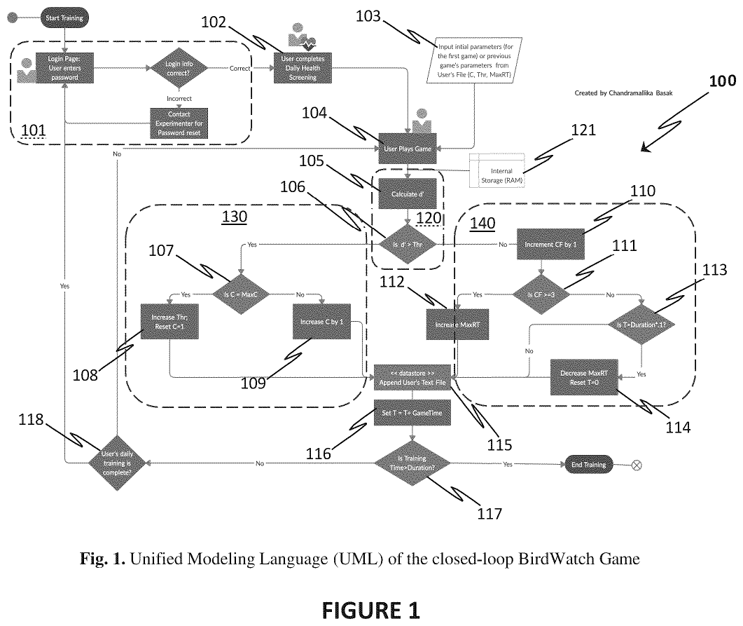

A detailed description of the BirdWatch Game Training is described in FIG. 1. In the UML, d′ is the memory discrimination accuracy from the previous block and MaxRT is the maximum allowed response time for the previous block.

The context index C is the number of contexts (that is trees in spatially distinct location) that vary from 1 to C (MaxC) and CF is Consecutive Failures where the participants failed to reach the threshold d′ₜ (Thr). A block is a unique instance of a game which comprises a set (or block) of trials. In an example embodiment, each block includes N trials, for example 80 trials (or events), where N birds appear in sequence (either in a predictable sequence or a random sequence). Each trial lasts for MaxRT minutes (example, 5 mins) at the beginning of training with MaxRT increasing as the individual progresses through the training.

Note: Please keep in mind that number of trials and MaxRT are variables in the program and can be adapted for neurodiversity across different ages. The novel program is nonverbal and therefore is amenable to both children (esp. those with Attention Deficit Hyperactive Disorder, Developmental Language Disorder) and well as older adults at risk of dementia. In the example embodiment, the number of trials and MaxRT have been tested on older adults (65-85 years of age).

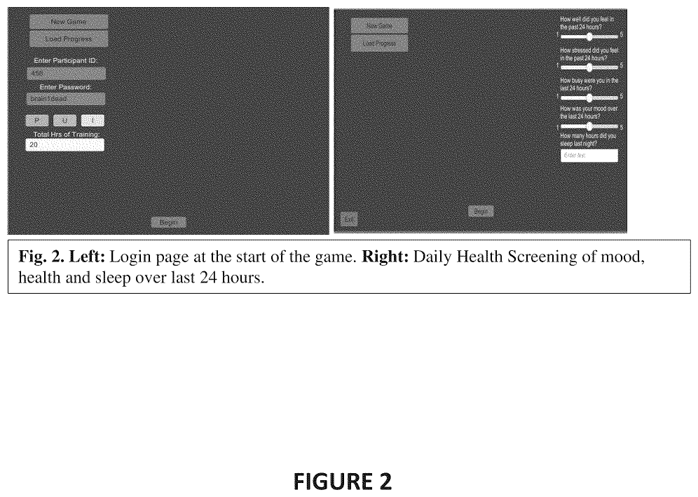

At the start of the training, at step 101, a login page appears (FIG. 2 Left), where the participant can enter their id and passwords (provided by us, thus ensuring that the data is complaint to the Health Insurance Portability and Accountability Act of 1996 (HIPAA).

After successful login, at step 102, a Daily Health Screening appears that asks you to drag a slider to report on a scale of 1 (low) to 5 (high) on:
1) How well did you feel in the past 24 hours?
2) How stressed did you feel in past 24 hours?
3) How busy were you in past 24 hours?
4) How was your mood in past 24 hours?
5) How many hours did you sleep last night?

For the final question, participants have to enter the number for the question below in the white box provided.

The game can start only after these values are entered, as after 10 days of game play, the game can be adjusted in terms of difficulty based on a weighted average of the metrics compared to their baseline over 30 days. This closed-loop design has not been utilized before in any cognitive training to our knowledge. The implementation of this design is described herein.

At step 103, initial parameters for the game are determined. The initial parameters may be set to a controlled extent by the user. The level of control of parameters or the predetermination of parameters may be set by a clinician. The hours one can play can be set by a clinician. The clinician can also set the game version that the participant will play: predictable version (Blue Button, stating “P”) or unpredictable version (Green Button, stating “U”) or a more complex version of the game (Yellow Button, stating “I”). For example, from our experience, it is recommended that people start by playing version P (5 hours over 2 weeks), followed by 2-4 weeks (>10 hours) of version U, followed by 4-8 (>15 hours) weeks of version I.

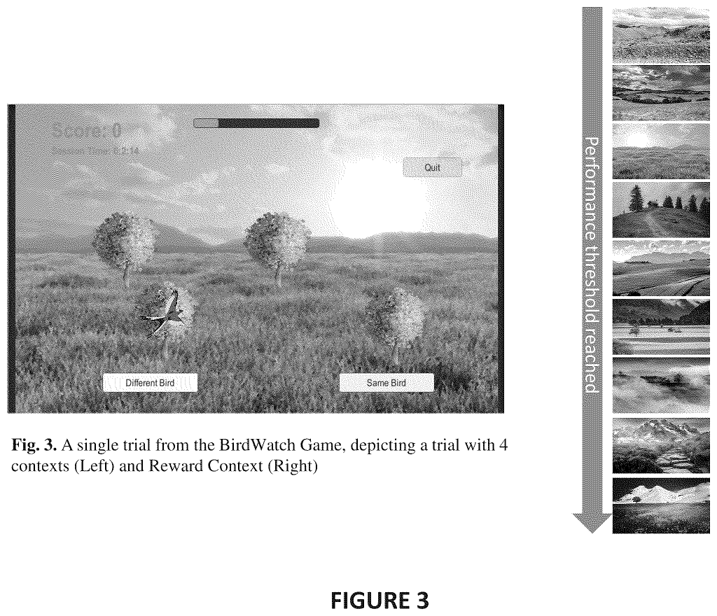

At step 104, the game is activated, and the user begins interacting within the game by responding within and completing a block of trials. In one embodiment, simplified renderings of birds were used for individual stimuli, with trees in spatially distinct locations utilized as contexts (see FIG. 3 Left). Both bird stimuli and tree contexts are displayed on a rendering of an outdoor scene, selected to be both aesthetically pleasing and to reinforce the narrative that the training task is a “Bird Watching Game”, as implied by the title of the task.

Additionally, a game-like player feedback was added to game in the form of a score display and a “reward” system. Score was calculated as follows:

$$Score = 100 ( Hit + CR ) - 50 ( Miss + F A ) + 1 \; 000 {d}^{′} ( 7 - M a x R T )$$

In the above equation, Hit is the total number of hits from the previous block, CR is the total number of correct rejections from the previous block, Miss is the total number of misses from the previous block, FA is the total number of false alarms from the previous block, d′ is the memory discrimination accuracy from the previous block and MaxRT is the maximum allowed response time for the previous block (see below). This score display was primarily implemented as an engagement tool which allowed participants to have a general sense of how their performance was progressing over time. However, it is also used as a performance metric to model learning rates of the participants as shown in Table 1 below.

A “reward” system was implemented by the “unlocking” of new background images as participants met performance milestones, specifically whenever performance threshold set by the program was increased (see FIG. 3 Right). This system is intended to reduce the monotony of performing the same task over multiple hours of training by periodically providing a different visual appearance over time, and to reinforce participant's success by tying this cosmetic change to performance milestones.

To further gamify this paradigm, we implemented BirdWatch Game within the Unity game engine (Version 2018.4.2f1, 2018), a robust game development toolkit commonly used in independent game development. This allows BirdWatch Game to be deployed and run across multiple electronic platforms (i.e. Windows computers, Android and Apple phones, etc.) as if it were a recreational video game. As an added benefit, the Unity engine is sufficiently feature-rich and expandable as to be comparable to data collection software more commonly used in cognitive science research (i.e. Eprime), which allowed for the collection of detailed performance metrics as described in the sections below.

Several methods of adjusting the difficulty of the BirdWatch Game based on the participant's real-time performance were implemented within the paradigm, based on past research which implicates individualized-adaptive training methodologies as efficacious (Brehmer, Westerberg, & Bäckman, 2012; Cuenen et al., 2016; Mihalca et al, 2011). Firstly, BirdWatch Game is capable of adjusting the number of contexts, C, utilized for a given block of trials based on participant performance in the recently completed (previous) block (see process 130).

Once the block of trials is complete in step 104, a skill check process 120 is performed where memory discrimination accuracy (d′) is calculated and compared to a threshold. Memory discrimination accuracy d′ is utilized as the measure of participant performance, and is calculated in step 105 for the completed (previous) block:

$${d}^{′} = z ( FA ) - z ( hit ) .$$

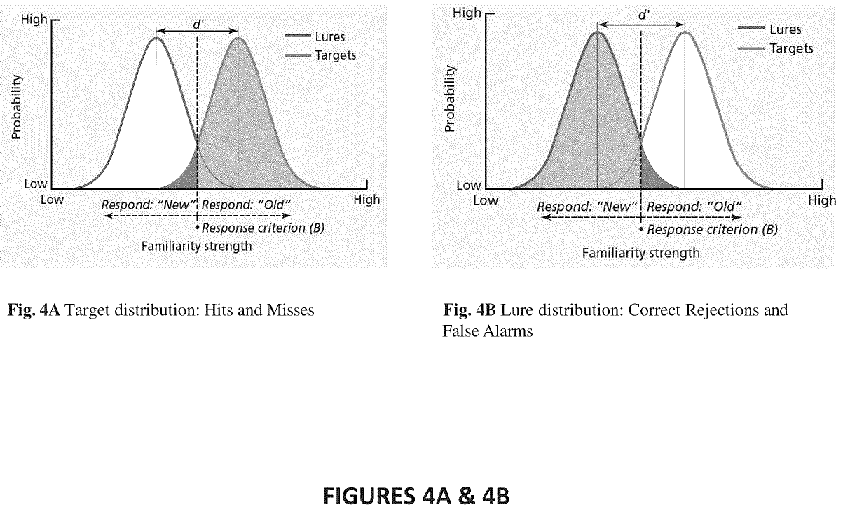

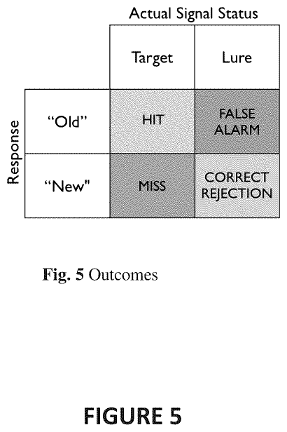

Referring to FIGS. 4 and 5, calculation of d′ in step 105 is described further. D-prime (d′) is a common measure of signal detection theory, which is the standardized difference between the signal present distribution and signal absent distribution (see FIGS. 4A and 4B). From context of this game, we need to first understand the types of signals present (target memory or lure) and the participant's response.

Thus, there are 4 possible outcomes (FIG. 5):

Two correct outcomes are:
Hits
   Correctly reporting the presence of the signal
Correct Rejections
   Correctly reporting the absence of the signal,

Two incorrect outcomes are:
False Alarms
   Incorrectly reporting presence of the signal when it did not occur
Misses
   Failing to report the presence of the signal when it occurred.

D prime (d′) over a block of N trials represents:
The distance between the signal present and signal absent distributions.
The participant's ability to discriminate the signal present and signal absent distributions.

Therefore, in calculating d′ of the previous block, FA is the number of false alarms from the previous block, and hit is the number of correct identifications made in the previous block, represented by the shaded green and shaded red in (FIGS. 4A and 4B). Thus, z(FA) and z(H) are the z-scores that correspond to the right-tail p-values represented by FA and Hit.

The 1/2N correction may be applied to account for floor and ceiling effects (Macmillan, & Creelman, 2005); for example, this correction is needed for adults with poor memory and who are susceptible to floor effects, such as older adults.

At step 106, the participant's d′ for the previous block is compared to a performance threshold, d′ₜ, and C is incremented by 1 for the next block if d′ is greater or equal to d′ₜ (See FIG. 1, step 120 Skill Check process). When d′ is greater or equal to d′ₜ then the game proceeds to step 107 of process 130. When d′ is less than d′ₜ then the game proceeds to step 110 of process 140.

Process 130 is a nested control of the game context. In one embodiment, the BirdWatch Game scales up to MaxC contexts (each context indicated by the context index C), given the size of the tablet used in Phase I clinical trial. However, this parameter of MaxC (set, for example, to be equal to six) is flexible based on screen and challenges desired by a participant based on their learning.

At step 107 of process 130, C is compared to MaxC. If C=MaxC contexts, and a participant performs above threshold (d′ₜ), the performance threshold is increased, and the number of contexts is reduced to one (step 108). This increase in d′ₜ is associated with the “reward system” with each increase in d′ₜ “unlocking” a new background display. In one embodiment, the performance threshold begins at 0.6, and increases by +0.2 for each participant success on an n=6 block, to a maximum of d′ₜ=3.

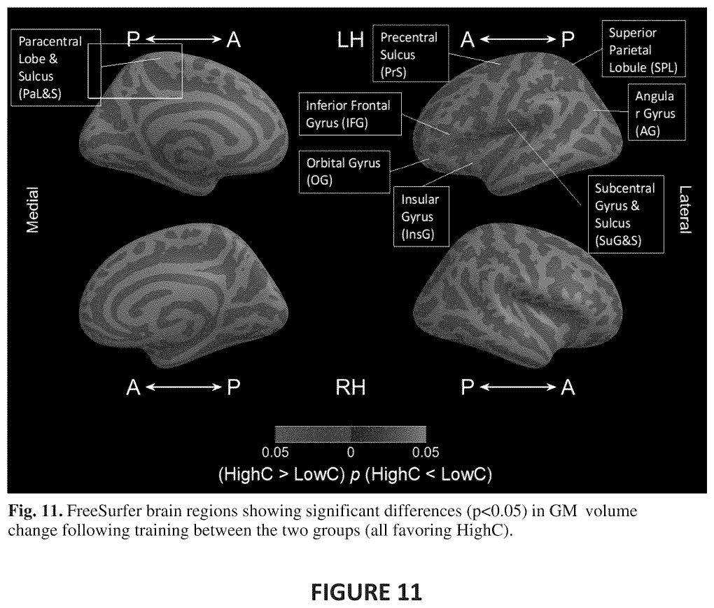

If the context index C has not reached MaxC while the participant performs above threshold (d′ₜ), then the context index C is increased by 1 at step 109. This system allows the BWGU paradigm to scale up difficulty in response to an individual participant's performance up to 72 times (6 contexts by 12 increases in threshold) over the course of training (see FIG. 11).

Additionally, the response time window in which a participant is able enter a response to the current stimuli also scales in two ways with participant's performance: via d′ₜ and MaxRT. This double-pronged approach has not been used before to our knowledge. In one embodiment, for example, participants have 5 seconds to respond to a new stimulus (i.e. MaxRT=5 s).

Turning to process 140, at step 110, when d′ is less than d′ₜ for any given block, a consecutive failure (CF) is logged by incrementing CF by 1. Then a test is applied at step 111, to determine if three consecutive failures across three consecutive blocks have occurred.

At step 113, for each 10% of the total expected training time T elapsed, MaxRT is decreased by 0.5 s to a minimum of 1 s. Conversely, at step 112, when three consecutive failures (CF) occur in three consecutive blocks, MaxRT is incremented by incremental time up to a maximum. For example, MaxRT is incremented by 0.5 s, to a maximum of 6 s. In other embodiments, the MaxRT decrement (or increment) may be different than 0.5 s and the minimum (maximum) of MaxRT may be different than 1 s (6s). In this way, time pressure is both increased and decreased in line with the participant's performance and progress through training. This flexible window of time either challenges a person to respond quickly if they are performing well or relax the time constraint of they are performing poorly. Such a feature which combines d′ increments as well as increment/decrement of response time window based on an individual's performance is novel in cognitive game training.

This novel dual-pronged approach comprises 1) the process 130 of first, systematically increasing the d′ₜ,, by nesting increasing C, from lower memory sensitivity (d′ₜ=0.6) to very high memory sensitivity (d′t=3), and 2) the process 140, of then systematically decreasing response time window (MaxRT) after the highest memory sensitivity is reached, targets memory sensitivity and response latency during memory updating in a nested closed-loop manner.

At step 115, the game variables including C, d′ₜ, T, and MaxRT and game results including trial-wise performance data are stored in internal storage 121. Trial-wise performance data collected by the program includes participant accuracy, reaction time, and trial characteristics (switch trial, update trial). Block-wise performance data collected includes Score, C, d′, and d′ₜ. At step 116, the training T is incremented and at step 117, the training time T is compared to a maximum duration of training. If complete, the training session ends and the user is logged out, otherwise the user continues to play in another block of trials at step 104.

In one embodiment, the BirdWatch Game was configured to administer continuous blocks of 80-trials each, with C, d′ₜ, (Thr) and MaxRT modulated between blocks as described. Between blocks, the BWGU training program pauses until the participant indicates they are ready to begin another block (at step 118) or chooses to exit the program. In the latter case, the current value of C, d′ₜ, and MaxRT, as well as the total training time completed, are saved by the program for use the next time the participant activates the training program. An additional feedback mechanism—a “progress bar”—was added to the BWGU training program to aid participants in tracking their progress through training. This progress bar, which can be seen in the top-center of FIG. 3, fills relative to the participant's progression through the assigned 20 hours of training, with the percentage of the bar filled reflecting the percentage of total training time elapsed.

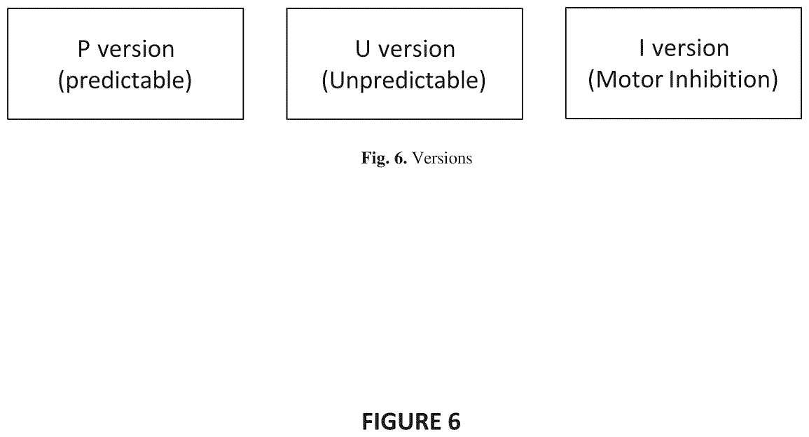

Referring to FIG. 6, in some embodiments there are multiple versions of the game available: the predictable (P) version, the unpredictable (U) version and the motor inhibition unpredictable (I) version.

While the game is being played (step 104), at any time you see only one bird on the screen.

Participants are instructed to determine if the bird that they are currently seeing matches the bird that had seen just before on the same tree. If they think that the birds are same, they press the “same” box, else the “different” box on the touchscreen of the tablet (FIG. 3 left). In predictable (P) version the birds appear in a predictable spatial order and, therefore, this aspect of the game is not necessarily innovative (although the closed loop individualized system is, as described before). Such predictable sequences have been used in non-individualized, standard, spatial n-back tasks (e.g., Verhaeghen, Cerella & Basak, 2004; Verhaeghen & Basak, 2005; Jaeggi et al., 2008). In such predictable sequences, the participant has to always switch their attention from one context to another. However, the novelty of the design comes from Unpredictable (U) and more complex motor inhibition (I) versions of the game.

In the new approach of the U version, the birds can appear in any one of the contexts (trees) unpredictably, such that for 50% of the trials there is no need to switch contexts (nonswitch trials) and for remaining 50% there is a need to switch between the contexts (switch trials). Such random switch and nonswitch manipulation within a block of trials necessitates greater cognitive control (Basak & Verhaeghen, 2011) than the predictable trials even in a n-back task. Functional magnetic resonance imaging (fMRI) data from our recently completed Phase I clinical trial suggest that the neural mechanisms of training and transfer from this unpredictable BirdWatch Game training in healthy aging may be a) increased engagement of the Central Executive Network (CEN) to exogenous cues, esp. left parietal and frontal regions, and b) deactivation of the Default Mode Network (DMN) that is responsible for lapses in attention to internalized endogenous cues. To our knowledge, there are no studies that have used this brain imaging evidence-based hypothesis to training older adults' cognition and brain functions. Moreover, no study uses unpredictably cued memory updating in a closed-loop training framework as it is an innovative line of research by me over past few years.

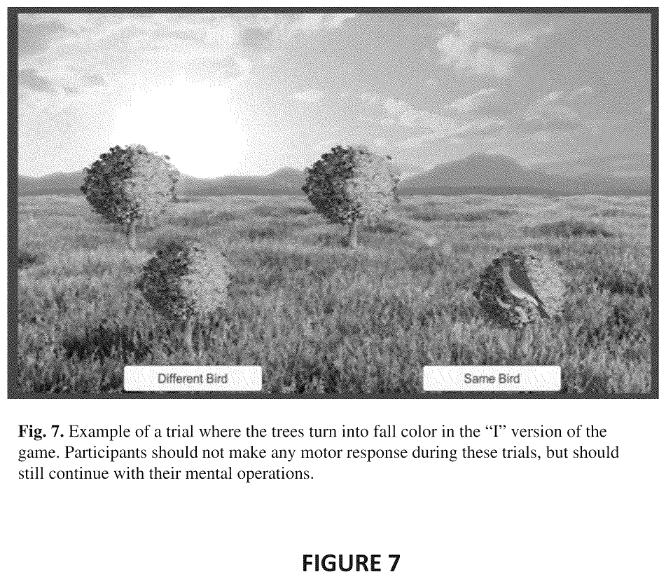

A more complex version of the BirdWatch game is conceived, called the motor inhibition unpredictable (I) version of the game (FIG. 3 left). This novel adaptation combines unpredictability of both endogenous and exogenous cues in memory. The birds appear in random contexts as in the U version of the game, thus necessitating greater deactivation of the DMN network. But for 10% of the trials in the I version, the trees turn into “fall” colors within 150 ms of their appearance (See FIG. 7); 150 ms is much earlier than a motor response after stimuli detection by a typical human can be made. In these trials, participants are asked to still continue with their mental operations that are required for subsequent comparisons, but to inhibit their motor responses for this trial. This motor-cognition dissociation can engage greater CEN activation to these exogenous visible cues. This innovation is new in the current version of the BirdWatch Game. The “I” version is an extension of “U” version—the birds do appear in unpredictable sequence as in “U” blocks, but for 10% of the trials in a block, participants have to withhold their motor response on the tablet but continue with the cognitive operations. The “I” approach builds on the “U” by adding a layer of difficulty, such that neural networks for motor and cognitive control are trained to dissociate for a few unpredictable trials. This can train the CEN and DMN brain networks more extensively than “U” version and represents another new approach to game training.

We conducted timeseries analysis of game learning and Daily Health Screening for participants in the Phase 1 Clinical Trial (Smith et al, 2022). To assess the individual-level influence of daily psychosocial factors on performance-over-time, we ran a series of auto-regressive integrated moving average (ARIMA) analyses using Simple Score as the dependent variable, Training Day as the indexing variable, and Wellness, Stress, Busyness, Mood, and Sleep as independent variables. This analysis was run independently for each participant, allowing for individual assessment of the impact of each moderator on performance over time. These ARIMA analyses were accomplished using the “forecast” package (Hyndman et al, 2021; Hyndman & Khandakar, 2008) for R (R Core Team, 2013). Instead of setting the AR, I, and MA, parameters of the ARIMA models a piori, the auto.arima function of the “forecast” package was used to procedurally select the ARIMA model that best fitted each participant's time-series. This auto-ARIMA approach examines all possible ARIMA models within the bounds specified, and selects a final model based on the Akaike Information Criterion (AIC), which is a model criterion that accounts for both goodness-of-fit and parsimony of the model (Akaike, 1973, 1987; Bozdogan, 1987, 2000; Sawa, 1978). Maximum parameter bounds for these auto-ARIMA analyses were set to AR<=5, I<=1, MA<=5.

ARIMA models were successfully fit for 34 participants. ARIMA models did not fit the remaining 3 participants due to a conjunction of low training time (all three participants discontinued the study prior to completing 5 hours of training) and a sparsity of daily survey responses. the value and significance of the psychosocial context moderators and sleep on each participants' performance-over-time also demonstrated notable heterogeneity. In total, 17 (50%) of the sample demonstrated performance-over-time which was demonstrably predicted by one or more of the examined psychosocial context variables and sleep, whereas the remaining 17 (50%) participants demonstrated no such relation. These results demonstrate a highly individualized effect of the examined psychosocial variables on training performance-over-time, including half of our sample for whom performance does not appear to be influenced by the psychosocial context variables examined.

In yet another aspect of the present invention, we have built in adaptability after 30 days of training that can account for the daily health screening variables.

In the daily health screening adaptivity, a first ARIMA model is calculated and fit to the 30 days of training data form each participant, and based on their model fit, will change the d′ₜ (Thr) for that day based on report of last 24 hours of mood and sleep metrics, with d′ₜ set to a lower threshold (by 0.2 units) than previous game settings if the scores are lower than their averaged past metrics, otherwise the game moves as expected with increase in dual-pronged challenges to d′ and MaxRT as described before.

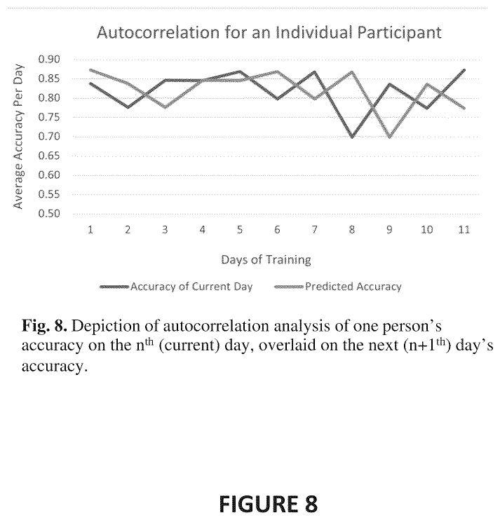

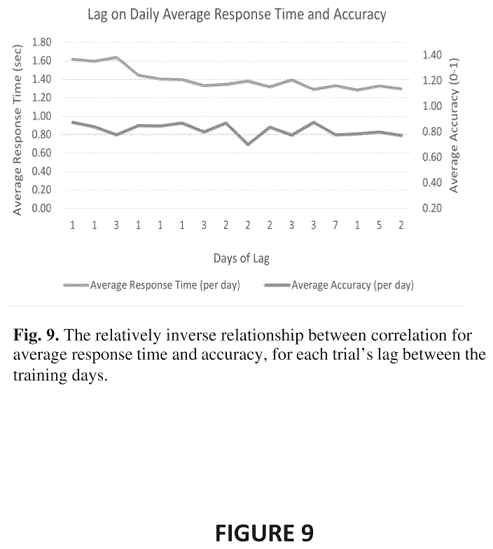

A detailed analysis of the effect of adherence on training performance was also conducted for this patent application. An autocorrelation analysis was performed on our pilot data of 49 subjects (including 12 new older adults to the Phase 1 clinical trial dataset) (FIG. 8). Adherence in this context is being measured by whether a participant is adhering to the prescribed training frequency (average lag between the training days). Greater adherence to training predicted better training outcomes. Specifically, the lag between sessions significantly predicted average daily accuracy (FIG. 9), suggesting that there could be learning benefits from shorter temporal distance between the training days.

In a recently completed NIH-funded Phase I clinical trial in old adults aged 65-80 years (NCT03988829), I recruited healthy older adults to test the feasibility and efficacy of the HighC BirdWatch training versus LowC BirdWatch Game training. In this trial, 28 older adults (NPRED=15, NUNP=13) completed all cognitive and neuroimaging assessments before and after 8 weeks of training. A group of young adults (N=24), who did not undergo any training, provided neuroimaging data for comparisons. Participants in both arms of BirdWatch Game Training were asked to train for 20 hours over a period of 8 weeks. Specifically, participants were asked to train for 2.5 hours each week, divided across two to three sessions. Training was performed at home using a 9.6′ Android tablet computer provided to the participants, with the BirdWatch Game Training training program pre-installed on that device.

Unpredictable HighC older adults had significantly large overall cognitive gains than predictable LowC group (p<. 01; Cohen's d=1.29), with greatest gains in executive control functions (p<. 01; Cohen's d=1.28) and processing speed (p=. 02; Cohen's d=0.97) constructs.

In this trial, adherence to this BirdWatch Game training was high, in spite of COVID-19 posing restrictions on contact with participants. As seen in Table 1 below, both Predictable (LowC) and Unpredictable (HighC) BirdWatch Game Training groups showed similar adherence to training (hours trained) and learning on the game (based on learning growth curve and highest level played).

TABLE 1
Mean (SD) of the two BirdWatch Game training
groups on adherence metrics and game learning
BirdWatch Game Adherence and Learning

| | LowC (Pred) | | HighC (Unp) | | | | |
|---|---|---|---|---|---|---|---|
| | Mean(SD) | | Mean(SD) | | t | df | Sig |
| Total Hours | 17.75 | (5.53) | 17.38 | (6.09) | 0.20 | 38 | 0.42 |
| Trained | | | | | | | |
| Highest Level | 52.1 | (15.68) | 47.35 | (21.77) | 0.79 | 38 | 0.22 |
| Played | | | | | | | |
| Learning | 679.32 | (356.3) | 550.6 | (335.89) | 1.18 | 38 | 0.12 |
| (Growth Rate) | | | | | | | |

We also conducted whole-brain MRI-BOLD analyses in this Phase I clinical trial that focused only on significant group by assessment interactions, cluster-corrected at Z>3.1, p<. 05. We evaluated changes in BOLD signals in the n-back task, with stimulus generalizability (Birds vs Digits) and cognitive control (2- and 3-back vs 0-back) as conditions of interest. The analyses resulted in two DMN clusters (Frontal Pole, Lingual Gyrus), where Unpredictable HighC showed significant deactivations at post-training than Predictable LowC for both trained (bird) and untrained (digits) stimuli, implicating generalizability of HighC training.

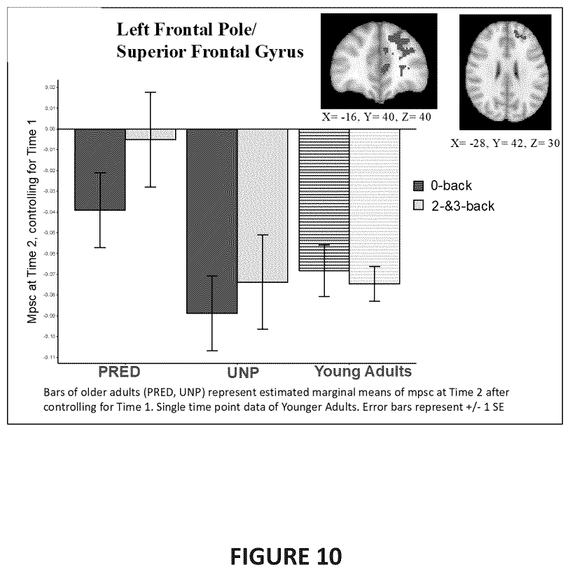

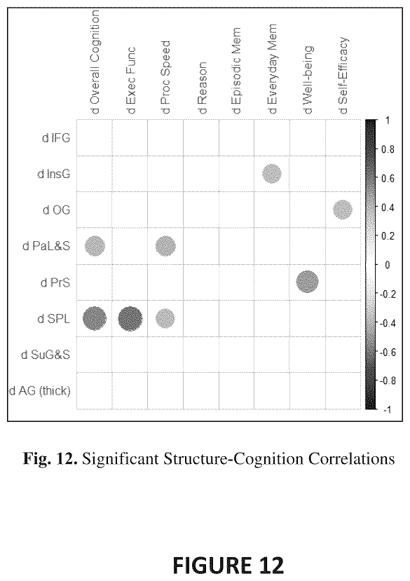

Moreover, old adults in HighC training were able to deactivate left frontal regions (e.g., frontal pole, FIG. 10) to the same extent as young adults, suggesting DMN deactivation as a mechanism for training-related cognitive gains. For the far transfer task (task-switching), HighC when compared to LowC resulted in post-training increases in neural efficiency matching that of young adults and in compensatory frontal BOLD signals. Regarding brain structure, maintenance of left lateral parietal and frontal gray matter volumes and cortical thickness of left parietal regions (FIG. 11) were found for HighC, but not for LowC who showed steady declines over the 10-12 weeks period. Increases in these gray matter volumes were correlated with gains in overall cognition, implicating the protective effects of these brain structures (FIG. 12).

As used herein, the phrase “at least one of,” when used with a list of items, means different combinations of one or more of the listed items can be used, and only one of each item in the list may be needed. In other words, “at least one of” means any combination of items and number of items may be used from the list, but not all of the items in the list are required. The item can be a particular object, a thing, or a category.

For example, without limitation, “at least one of item A, item B, or item C” may include item A, item A and item B, or item B. This example also may include item A, item B, and item C or item B and item C. Of course, any combination of these items can be present. In some illustrative examples, “at least one of” can be, for example, without limitation, two of item A; one of item B; and ten of item C; four of item B and seven of item C; or other suitable combinations.

As used herein, “a number of” when used with reference to items, means one or more items. For example, “a number of different types of networks” is one or more different types of networks. In illustrative example, a “set of” as used with reference items means one or more items. For example, a set of metrics is one or more of the metrics.

The description of the different illustrative embodiments has been presented for purposes of illustration and description and is not intended to be exhaustive or limited to the embodiments in the form disclosed. The different illustrative examples describe components that perform actions or operations. In an illustrative embodiment, a component can be configured to perform the action or operation described. For example, the component can have a configuration or design for a structure that provides the component an ability to perform the action or operation that is described in the illustrative examples as being performed by the component. Further, to the extent that terms “includes”, “including”, “has”, “contains”, and variants thereof are used herein, such terms are intended to be inclusive in a manner similar to the term “comprises” as an open transition word without precluding any additional or other elements.

Many modifications and variations will be apparent to those of ordinary skill in the art. Further, different illustrative embodiments may provide different features as compared to other desirable embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

What is claimed is:

1. A computer-implemented neurocognitive training game, comprising:
presenting, in a user interface, a number of questions regarding different health parameters of a user within a previous 24 hour period;
responsive to entry of answers to all of the questions, presenting, in the user interface, a number of trials, wherein each trial has a specified response time window, and wherein each trial comprises a number of images displayed within spatially distinct contexts;
generating a user score based on the correct identification of the images in the trials by the user; and
adjusting difficulty and the response time window for the trials according to the user score.
